(19) United States
(12) Patent Application Publication
(10) Pub. No.: US 2022/0068476 A1
(43) Pub. Date: Mar. 3, 2022

(54) RESAMPLING EEG TRIAL DATA

(71) Applicant: X Development LLC, Mountain View, CA (US)

(72) Inventors: Katherine Elise Link, Palo Alto, CA (US); Vladimir Miskovic, Binghamton, NY (US); Nina Thigpen, Sunnyvale, CA (US); Mustafa Ispir, Mountain View, CA (US); Garrett Raymond Honke, Mountain View, CA (US); Pramod Gupta, Mountain View, CA (US)

(21) Appl. No.: 17/007,193

(22) Filed: Aug. 31, 2020

(51) Int. Cl. G16H 50/20; G06N 3/08; G06N 7/00

(52) U.S. Cl. CPC G16H 50/20; G06N 7/005; G06N 3/08

(57) ABSTRACT

Systems and processes described herein can expand a limited data set of EEG trials into a larger data set by resampling subsets of EEG trial data. Implementations may employ one or more of a variety of different resampling techniques. For example, a subset of the available training data is selected to form a new set of training data. The subset can be selected using replacement (e.g., a sample can be selected more than once, and thus represented multiple times in the new set of training data). Alternatively the subset can be selected without using replacement (e.g., each sample is able to be selected only once, and thus represented a maximum of one time in the new set of training data).

### TECHNICAL FIELD

This disclosure generally relates to using resampling methods to enhance training data for a machine learning model.

### BACKGROUND

In some machine learning processes, a limited amount of EEG data is available for each individual. Machine learning models typically perform better when trained on large datasets. Therefore, a method for generating additional EEG trials is for training is desired.

### SUMMARY

In general, the disclosure relates to data augmentation for training data that includes aggregated electroencephalogram (EEG) trials for use in training a machine learning model to assess the mental health in individuals. In some cases, multiple EEGs are aggregated into a single representative EEG trial for each individual. This results in a single data point per individual, which can be inadequate for training a machine learning model unless a large number of individuals are available. In order to more effectively use the training data that is available, it can be resampled to create additional training data.

In general, innovative aspects of the subject matter described in this specification can be embodied in systems, methods, and non-transitory storage media which perform operations including: identifying training data including a plurality of embeddings, each embedding representing EEG trial data for a particular individual. The method can then select, from the training data and for inclusion in one or more subsets of the training data, particular embeddings based on a random probability distribution that is associated with a weighting factor assigned to each embedding. An augmented set of training data is generated by combining two or more subsets, each augmented set of training data including more embeddings than the training data. The augmented set of training data is provided as training input to a machine learning model. These and other implementations can each optionally include one or more of the following features.

In some implementations, the weighting factor is assigned to each embedding based on a determined trial quality for each embedding. Trial quality can be determined by analyzing the trial data using an autoencoder network or a convolutional neural network.

In some implementations, the random probability distribution is a uniform probability distribution.

In some implementations, the random probability distribution is a Gaussian probability distribution.

In some implementations, after selecting an embedding for inclusion in one of the subsets, the embedding is permitted to be potentially selected again in the same subset.

In some implementations, after selecting an embedding for inclusion in one of the subsets, the embedding is prevented from being selected again in the same subset.

In some implementations, providing the augmented set of training data includes providing a first portion of the augmented set and performing a first training of the machine learning model, and providing a second portion of the augmented data set and performing a second training of the machine learning model.

The details of one or more implementations of the subject matter described in this disclosure are set forth in the accompanying drawings and the description below. Other features, aspects, and advantages of the subject matter will become apparent from the description, the drawings, and the claims.

### DESCRIPTION OF DRAWINGS

FIG. 1 depicts an example of a system architecture for EEG analysis.

FIG. 2 depicts a detailed diagram describing an example resampling method.

FIG. 3 is a flow diagram depicting an example method for resampling EEG data to be used in machine learning models.

FIG. 4 is a schematic diagram of a computer system.

### DETAILED DESCRIPTION

The disclosure generally relates to data augmentation for training data that includes aggregated electroencephalogram (EEG) trials for use in training a machine learning model to assess the mental health in individuals. In some cases, multiple EEGs are aggregated into a single representative EEG trial for each individual. This results in a single data point per individual, which can be inadequate for training a machine learning model unless a large number of individuals are available. In order to more effectively use the training data that is available, it can be resampled to create additional training data.

Systems and processes described herein can expand a limited data set of EEG trials into a larger data set by resampling subsets of EEG trial data. Implementations may employ one or more of a variety of different resampling techniques. For example, a subset of the available training data is selected to form a new set of training data. The subset can be selected using replacement (e.g., a sample can be selected more than once, and thus represented multiple times in the new set of training data). Alternatively the subset can be selected without using replacement (e.g., each sample is able to be selected only once, and thus represented a maximum of one time in the new set of training data). In some implementations, sampling the available training data is performed using a predefined method. For example, if three new data sets are to be generated, the first new data set can include data points 1, 4, 7, etc. The second data set can include data points 2, 5, 8, etc. The third data set can include data points 3, 6, 9, and so on. In some implementations data is selected from the available training data in a random fashion (e.g., with a random number generator, or a pseudo-random selection). In some implementations, new data points are selected randomly yet with an associated probability factor. The probability factor can be calculated based on a “data quality” determination, such that samples with a higher data quality have a larger probability of being selected than samples with a lower data quality. The data quality determination can be made based on statistical analysis of the data (e.g., standard deviation, or a Fourier transform analysis, among others). In some implementations the data quality is determined using a machine learning model.

Resampling as described allows a relatively small set of training data to be used to train a machine learning model, without causing the machine learning model to over-fit, or be overly dependent on the training data. For example, an outlier in the training data may have a reduced impact when the machine learning model is trained with resampled training data, then it would if it were trained directly on the available data.

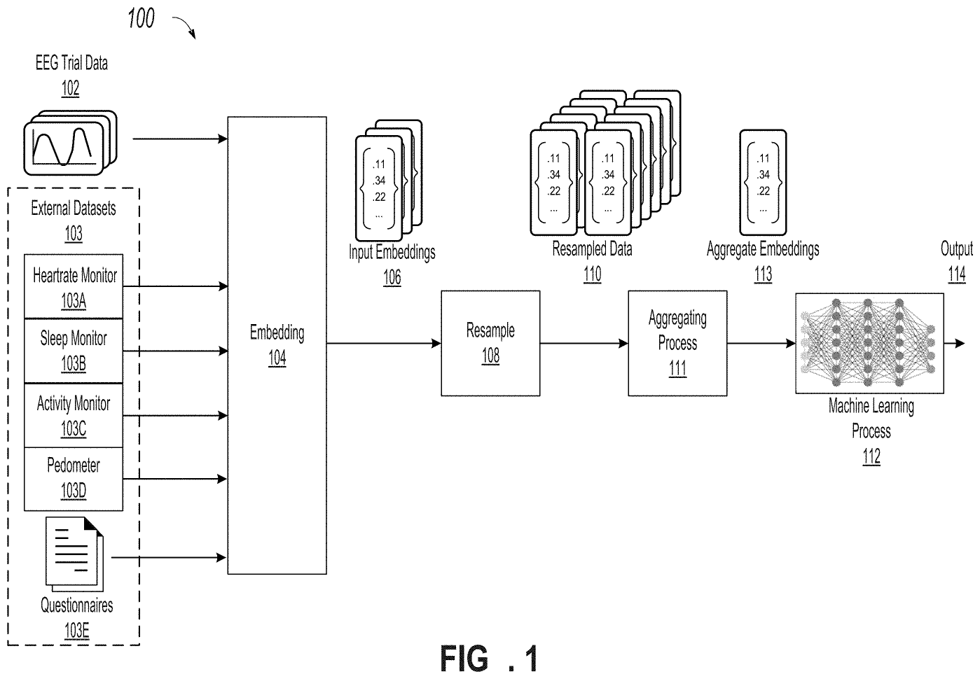

FIG. 1 depicts an overall system architecture for EEG analysis. The system 100 receives EEG trial data 102 which can be digital representations of analog measurements taken during an EEG trial during which an individual is presented with various stimulus. For example, stimulus intended to trigger particular responses in portions of the brain, such as the visual cortical system, or the anterior cingulate cortex, can be presented to an individual and the corresponding brainwave response recorded in the EEG trial can be marked or labeled, and associated with a timestamp of when the stimulus was presented. Each set of EEG trial data 102 can be provided as input to an embedding process 104. The stimulus can include, but is not limited to, visual content such as images or video, audio content, interactive content such as a game, or a combination thereof. For example, emotional content (e.g., a crying baby; a happy family) can be configured to probe the brain's response to emotional images. As another example, visual attentive content can be configured to measure the brain's response to the presentation of visual stimuli. Visual attentive content can include, e.g., the presentation of a series of images that change between generally positive or neutral images and negative or alarming images. For example, a set of positive/neutral images (e.g., images of a stapler, glass, paper, pen, glasses, etc.) can be presented with a negative/alarming images (e.g., a frightening image) interspersed there between. The images can be presented randomly or in a pre-selected sequence. Moreover, the images can alternate or “flicker” at a predefined rate. As another example, error monitoring content can be used to measure the brain's response to making mistakes. Error monitoring content can include, but is not limited to, interactive content designed to elicit decisions from an individual in a manner that is likely to result in erroneous decisions. For example, the interactive content can include a test using images of arrows and require the individual to select which direction the arrow(s) is/are pointing, but may require the decisions to be made quickly so that the user will make errors. In some implementations, no content is presented, e.g., in order to measure the brain's resting state to obtain resting state brainwaves.

External datasets 103 are datasets that are separate from the EEG trial data. They can include, but are not limited to a data recorded from a heartrate monitor 103A, a sleep monitor 103B, an activity monitor 103C, a pedometer 103D, and one or more questionnaires 103E. The external datasets can, in some cases, be collected in between EEG trials where EEG trial data 102 is collected. For example, if an individual receives an EEG trial once per week, they can additionally be asked to wear a sleep monitor every night in between trials. In some implementations external datasets 103 can be collected simultaneously with EEG trial data 102. For example, when an EEG trial is occurring, the individual can wear a heartrate monitor providing heartrate data 103A during the trial. This heartrate data 103A can largely correlate to a particular set of EEG trial data 102, and can show information corresponding to certain stimulus provided to the individual during an EEG trial. In some implementations, individuals can use a wearable data collection device (e.g., a smart watch, or other necklace or other wearable device) which collects data (e.g., sleep, activity, heartrate etc.) continuously for a period of time (e.g., days, weeks, etc.). Additionally individuals can complete surveys or questionnaires 103E periodically (e.g., daily) designed to determine information about their current mental state.

The embedding process 104 converts the raw EEG trial data 102 and the external datasets 103 into vectors of fixed length, resulting in an input embedding 106 for each set of EEG trial data 102 and each external dataset 103. In some implementations, the embedding process 104 is a convolutional neural network (CNN) that is trained simultaneously with the rest of the neural networks in system 100. The embedding process 104 can accept analog or digital data from each set of EEG trial data 102, as well as additional data such as metadata (e.g., timestamps, manual data tagging, etc.). In some implementations, the embedding process 104 is a part of an upstream CNN performing additional or external analysis on the EEG trial data 102 and external datasets 103. In these implementations, while the final or output layer of the upstream CNN can be used for separate analysis, each neuron in the penultimate layer of the CNN is used in the embedding process 104. These neurons each have a value which can be mapped to a vector representing the input embedding 106 which is to be the output of the embedding process 104. In some implementations, data from each source (e.g., EEG trial data 102, and external datasets 103) are embedded separately, using unique processes. For example, heartrate data can be embedded using a time sampled version of the analog heartrate data.

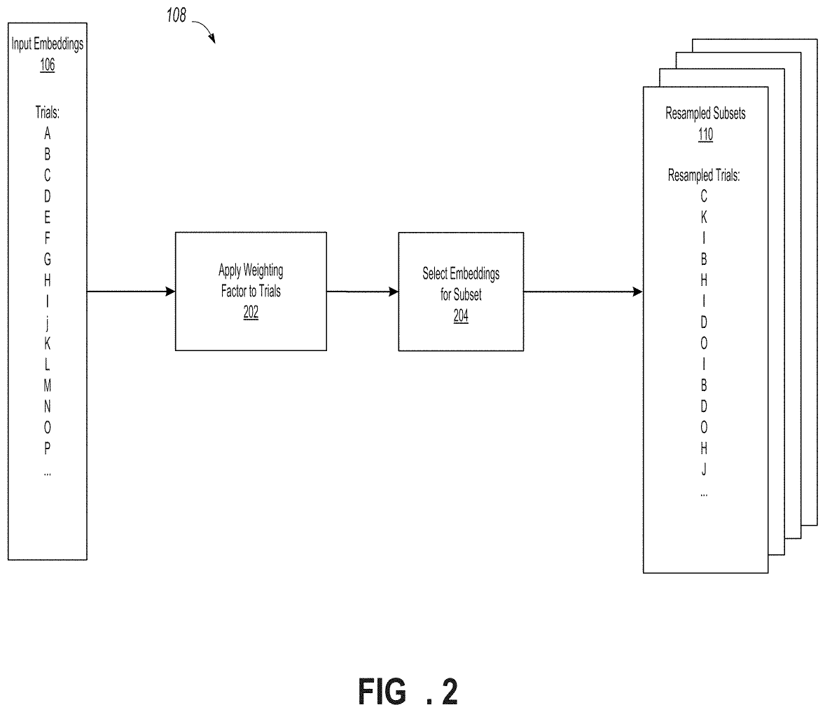

Multiple input embeddings 106 can then be provided to a resampling process 108. In some implementations, each individual will have an associated data corpus with a number embeddings representing EEG trials data 102, and external datasets 103. The resampling process 108 can generate a set of resampled data 110. This process is described in further detail with respect to FIG. 2.

The resampled data 110 or a subset of the resampled data 110 can then be aggregated by an aggregating process 111. The aggregating process can take resampled data 110 and combine it to generate an aggregate embedding to be used by a machine learning model. In some implementations, the input embeddings 106 are resampled by the resampling process 108 in order to generate several subsets of resampled data 110. This allows the aggregating process 111 to generate several aggregate embeddings 113, one for each subset, which can then be used to train downstream machine learning models (e.g., machine learning process 112). The aggregating process 111 can be an averaging, or a weighted average of the resampled data 110. In some implementations, the aggregating process 111 uses a neural network to determine how best to generate an aggregate embedding which is representative of the resampled data 110 to be aggregated, retaining relevant information while filtering out noise. For example, the aggregating process can be all or a portion of the transformer neural network, which uses a self-attention algorithm to identify portions of its input which should be retained.

The aggregate embeddings 113 can then be used for further analysis/classification, or for training e.g., a classification neural network. In some implementations, the machine learning process 112 can be a feedforward autoencoder neural network. For example, the machine learning process 112 can be a three-layer autoencoder neural network. The machine learning process 112 may include an input layer, a hidden layer, and an output layer. In some implementations, the neural network has no recurrent connections between layers. Each layer of the neural network may be fully connected to the next, e.g., there may be no pruning between the layers. The machine learning process 112 can include an optimizer for training the network and computing updated layer weights, such as, but not limited to, ADAM, Adagrad, Adadelta, RMSprop, Stochastic Gradient Descent (SGD), or SGD with momentum. In some implementations, the machine learning process 112 may apply a mathematical transformation, e.g., a convolutional transformation or factor analysis to input data prior to feeding the input data to the network.

In some implementations, the machine learning process 112 can be a supervised model. For example, for each input provided to the model during training, the machine learning process 112 can be instructed as to what the correct output should be. The machine learning process 112 can use batch training, e.g., training on a subset of examples before each adjustment, instead of the entire available set of examples. This may improve the efficiency of training the model and may improve the generalizability of the model. The machine learning process 112 may use folded cross-validation. For example, some fraction (the “fold”) of the data available for training can be left out of training and used in a later testing phase to confirm how well the model generalizes. In some implementations, the machine learning process 112 may be an unsupervised model. For example, the model may adjust itself based on mathematical distances between examples rather than based on feedback on its performance.

In some examples, the machine learning process 112 can provide a binary output label 114, e.g., a yes or no indication of whether the individual is likely to have a particular mental disorder. In some examples, the machine learning process 112 provides a score output 114 indicating a likelihood that the individual has one or more particular mental conditions. In some examples, the machine learning process 112 can provide a severity score indicating how severe the predicted mental condition is likely to be. In some implementations, the machine learning process 112 sends output data indicating the individual's likelihood of experiencing a particular mental condition to a user computing device. For example, the machine learning process 112 can send its output to a user computing device associated with the individual's doctor, nurse, or other case worker.

FIG. 2 is a diagram illustrating an example method for resampling that can be employed by the resampling process 108 of FIG. 1. The resampling process 108 receives input embeddings 106 which can include a multitude of trials. In some implementations, the input embeddings 106 also include data from external sources, such as external datasets 103 as discussed with respect to FIG. 1.

At 202 a weighting factor is applied to each trial in the set of input embeddings 106. The weighting factor is used to determine a probability of selection for a resampled subset. In some implementations, there is no weighting factor, or the weighting factor is the same for each trial, resulting in a uniform distribution and equal probability for each trial to be selected. In some implementations, the weighting factor is based on a determined trial quality for each trial, with trials of higher quality being given a larger weighting factor, and therefore more likely to be selected for resampling. In some implementations, trial quality is determined for each trial, based on a statistical analysis of the data in the trial.

Statistical analysis can include, for example determining a signal to noise ratio by performing a noise quantization and measuring signal amplitude. Additional statistical methods can be used, for example, correlation, root mean square error (RMSE) analysis, entropy analysis or others. In some implementations, trial quality can be determined using an autoencoder network. The autoencoder network can receive a trial as input, encoding it into a low dimensional representation, which can be analyzed using, for example, statistical methods, to determine a trial quality. In some implementations, a convolutional neural network is used to determine trial quality. The convolutional neural network can be trained to recognize patterns and data that correlate with high quality trials, and provide an accurate trial quality assessment for an input trial. In some implementations, trial quality is used to sort the trials in the input embeddings 106, then weighting factors are applied to each trial based on a standard probability distribution (e.g., a Gaussian distribution, or a geometric distribution, etc.).

Once a weighting factor is determined for each trial, trials are selected at 204 for a subset to form a resampled subset 110. The trials can be selected based on their weighting factor. For example, a trial with a weighting factor of 4 might be twice as likely to be selected as a trial with a weighting factor of 2. The trials can be selected using, for example, a pseudo random number generator. In some implementations, the selection is done with replacement. In other words, if a given trial is selected, it has an equal chance of being selected again for the same resampled subset. In some implementations, the selection is done without replacement, or such that once a particular trial is selected, it cannot be selected again to for this resampled subset 110. In some implementations, the selection is made with replacement, however the weighting factor for each selected trial is modified following selection, reducing its likelihood of being selected again.

The result of the selection process is one or more resampled subsets 110. Each resampled subset 110 can be made up of some proportion (e.g., 60%, or 90%, etc.) of the original input embeddings 106. It is possible to generate a several resampled subsets 110 from a single set of input embeddings 106. In this manner, the resampled subsets 110 represent additional data to be further processed for training a machine learning model. In some implementations, the resampled subsets 110 can be used directly as input to an already trained machine learning model, in order to have the machine learning model provide multiple results, allowing for statistical or probability analysis of the results, instead of yielding a single result.

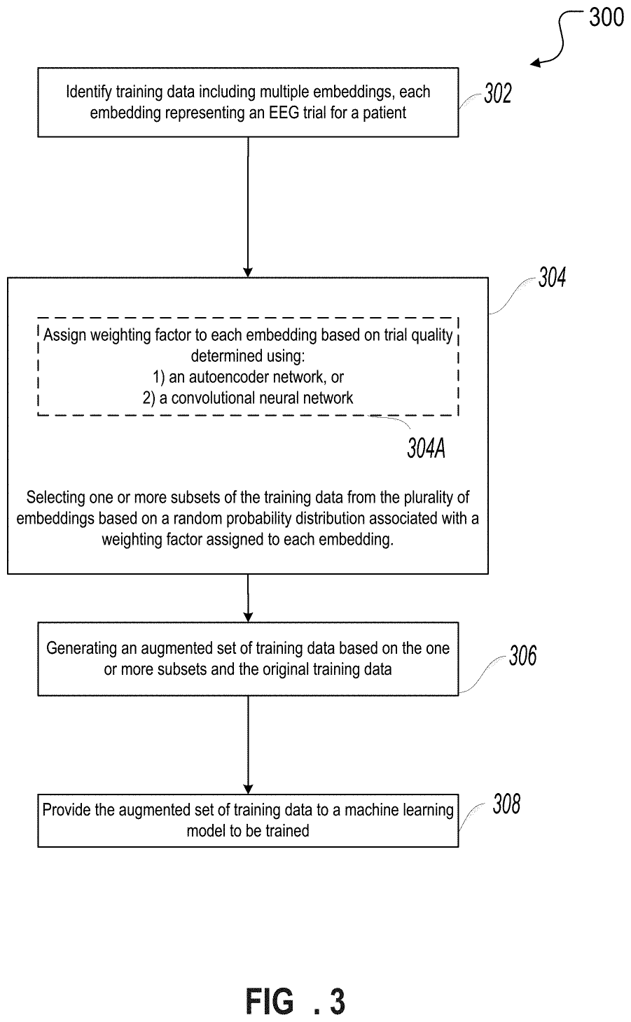

FIG. 3 is a flow diagram of an example process 300 for resampling EEG trial embeddings and external embeddings. It will be understood that process 300 may be performed, for example, by any suitable system, environment, software, and hardware, or a combination of systems, environments, software, and hardware as appropriate. In some instances, process 300 can be performed by the system as described in FIG. 1, or portions thereof, and further described in FIG. 2, as well as other components or functionality described in other portions of this description. In other instances, process 300 may be performed by a plurality of connected components or systems. Any suitable system(s), architecture(s), or application(s) can be used to perform the illustrated operations.

At 302, training data including a plurality of embeddings representing a plurality of EEG trials for a particular individual are determined. The plurality of embeddings can each represent a portion or segment of EEG data, or an entire EEG trial, including labeled stimulus presented during the trial.

At 304, one or more subsets of the training data are selected based on a random probability distribution associated with a weighting factor assigned to each embedding. The weighting factor can be assigned randomly, or in some cases uniformly (e.g., a weighting factor of 1 can be used for all embeddings). In some implementations the weighting factors are assigned based on a standard probability distribution (e.g., a Gaussian distribution, or a geometric distribution, etc.). Optionally, at 304A, a weighting factor can be assigned to each embedding based on a determined trial quality, where the trial quality is determined using an autoencoder network, or a convolutional neural network.

At 306, an augmented set of training data is generating by combining two or more of the selected subsets of the training data. In some implementations, the combined training data is all provided as a single large augmented set of data. In some implementations, multiple augmented training sets are generated, and training can be performed sequentially, using multiple augmented sets.

At 308, the augmented set of training data is provided to a machine learning model in order to train the machine learning model. For example, the augmented set of training data can be applied as batch training data to the machine learning model, e.g., the model can be trained on a subsets of the training data before each adjustment, instead of the entire available set of training data. In some implementations, the machine learning model can be trained using folded cross-validation. For example, some fraction (the “fold”) of the augmented set of training data can be left out of training and used in a later testing phase to confirm how well the model generalizes. In some implementations, the machine learning process 112 may be an unsupervised model. For example, the model may adjust itself based on mathematical distances between training data in the augmented set of training data rather than based on feedback on its performance.

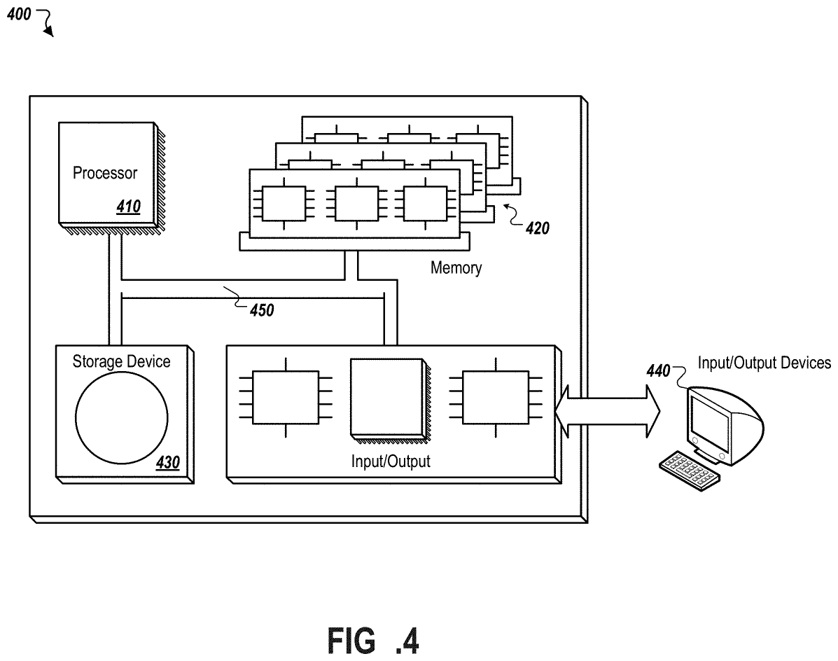

FIG. 4 is a schematic diagram of a computer system 400. The system 400 can be used to carry out the operations described in association with any of the computer-implemented methods described previously, according to some implementations. In some implementations, computing systems and devices and the functional operations described in this specification can be implemented in digital electronic circuitry, in tangibly-embodied computer software or firmware, in computer hardware, including the structures disclosed in this specification (e.g., system 400) and their structural equivalents, or in combinations of one or more of them. The system 400 is intended to include various forms of digital computers, such as laptops, desktops, workstations, personal digital assistants, servers, blade servers, mainframes, and other appropriate computers, including vehicles installed on base units or pod units of modular vehicles. The system 400 can also include mobile devices, such as personal digital assistants, cellular telephones, smartphones, and similar computing devices. Additionally, the system can include portable storage media, such as, Universal Serial Bus (USB) flash drives. For example, the USB flash drives may store operating systems and other applications. The USB flash drives can include input/output components, such as a wireless transducer or USB connector that may be inserted into a USB port of another computing device.

The system 400 includes a processor 410, a memory 420, a storage device 430, and an input/output device 440. Each of the components 410, 420, 430, and 440 are interconnected using a system bus 450. The processor 410 is capable of processing instructions for execution within the system 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the system 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the system 400. In one implementation, the storage device 430 is a computer-readable medium. In various different implementations, the storage device 430 may be a floppy disk device, a hard disk device, an optical disk device, or a tape device.

The input/output device 440 provides input/output operations for the system 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Further to the descriptions above, a user may be provided with controls allowing the user to make an election as to both if and when systems, programs, or features described herein may enable collection of user information. In addition, certain data may be treated in one or more ways before it is stored or used, so that personally identifiable information is removed. For example, a user's identity may be treated so that no personally identifiable information can be determined for the user, or a user's skin data and/or diagnosis cannot be identified as being associated with the user. Thus, the user may have control over what information is collected about the user and how that information is used

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A computer-implemented method executed by one or more processors and comprising:
identifying training data comprising a plurality of embeddings, wherein each embedding represents EEG trial data for a particular individual;
selecting, from the training data and for inclusion in one or more subsets of the training data, particular embeddings based on a random probability distribution associated with a weighting factor assigned to each embedding;
generating an augmented set of training data by combining two or more subsets, wherein the augmented set of training data comprises more embeddings than the training data; and
providing the augmented set of training data as training input to a machine learning model.

2. The method of claim 1, wherein the weighting factor assigned to each embedding is determined based on determining a trial quality for each embedding, wherein the trial quality is determined by:
analyzing the EEG trial data using at least one of an autoencoder network or a convolutional neural network.

3. The method of claim 1, wherein the random probability distribution is a uniform probability distribution.

4. The method of claim 1, wherein the random probability distribution is a Gaussian probability distribution.

5. The method of claim 1, further comprising after selecting an embedding for inclusion in one of the subsets, permitting the embedding to be potentially selected again in the same subset.

6. The method of claim 1, further comprising after selecting each embedding in the one or more subsets, preventing the embedding from being selected again in the same subset.

7. The method of claim 1, wherein providing the augmented set of training data comprises providing a first portion of the augmented set and performing a first training of the machine learning model, and providing a second portion of the augmented set and performing a second training of the machine learning model.

8. A system, comprising:
one or more processors;
one or more tangible, non-transitory media operably connectable to the one or more processors and storing instructions that, when executed, cause the one or more processors to perform operations comprising:
identifying training data comprising a plurality of embeddings, wherein each embedding represents EEG trial data for a particular individual;
selecting, from the training data and for inclusion in one or more subsets of the training data, particular embeddings based on a random probability distribution associated with a weighting factor assigned to each embedding;
generating an augmented set of training data by combining two or more subsets, wherein the augmented set of training data comprises more embeddings than the training data; and
providing the augmented set of training data as training input to a machine learning model.

9. The system of claim 8, wherein the weighting factor assigned to each embedding is determined based on determining a trial quality for each embedding, wherein the trial quality is determined by:
analyzing the trial data using at least one of an autoencoder network or a convolutional neural network.

10. The system of claim 8, wherein the random probability distribution is a uniform probability distribution.

11. The system of claim 8, wherein the random probability distribution is a Gaussian probability distribution.

12. The system of claim 8, further comprising after selecting an embedding for inclusion in one of the subsets, permitting the embedding to be potentially selected again in the same subset.

13. The system of claim 8, further comprising after selecting each embedding in the one or more subsets, preventing the embedding from being selected again in the same subset.

14. The system of claim 8, wherein providing the augmented set of training data comprises providing a first portion of the augmented set and performing a first training of the machine learning model, and providing a second portion of the augmented set and performing a second training of the machine learning model.

15. A non-transitory computer readable storage medium storing instructions that, when executed by at least one processor, cause the at least one processor to perform operations comprising:
identifying training data comprising a plurality of embeddings, wherein each embedding represents EEG trial data for a particular individual;
selecting, from the training data and for inclusion in one or more subsets of the training data, particular embeddings based on a random probability distribution associated with a weighting factor assigned to each embedding;
generating an augmented set of training data by combining two or more subsets, wherein the augmented set of training data comprises more embeddings than the training data; and
providing the augmented set of training data as training input to a machine learning model.

16. The medium of claim 15, wherein the weighting factor assigned to each embedding is determined based on determining a trial quality for each embedding, wherein the trial quality is determined by:
analyzing the trial data using at least one of an autoencoder network or a convolutional neural network.

17. The medium of claim 15, wherein the random probability distribution is a uniform probability distribution.

18. The medium of claim 15, wherein the random probability distribution is a Gaussian probability distribution.

19. The medium of claim 15, further comprising after selecting an embedding for inclusion in one of the subsets, permitting the embedding to be potentially selected again in the same subset.

20. The medium of claim 15, further comprising after selecting each embedding in the one or more subsets, preventing the embedding from being selected again in the same subset.
